(19) United States
(12) Patent Application Publication
(10) Pub. No.: US 2016/0374846 A1
(43) Pub. Date: Dec. 29, 2016

(54) Rhinoplasty Cotrol Molds (transverse, Axial, Basal, Final, Final Splint)

(71) Applicant: Seyed Soroush Valinia, Tehran (IR)

(72) Inventor: Seyed Soroush Valinia, Tehran (IR)

(73) Assignee: Seyed Soroush Valinia, Tehran (IR)

(21) Appl. No.: 15/179,982

(22) Filed: Jun. 11, 2016

Related U.S. Application Data
(60) Provisional application No. 62/246,635, filed on Oct. 27, 2015.

(30) Foreign Application Priority Data
Jun. 26, 2015 (IR) 13945014000300332
Jun. 6, 2016 (IB) PCT/IB2016/053304

(51) Int. Cl. A61F 5/08

(52) U.S. Cl. CPC A61F 5/08

(57) ABSTRACT

In most rhinoplasty cases surgeon should control whether different parts of Applicant's nose is formed well and refine disfigurements. Since surgeons have no instrument to measure changes during the surgery, they have to use their bare eye to decide whether to modify the configuration or not, which can cause the result to be different from what is contemplated by applicant. I designed 5 custom control molds to control correctness of different operation steps during the surgery to minimize surgeon's errors. Using these control molds enables surgeon to decide whether to keep modifying different part of nose in order to achieve best result.

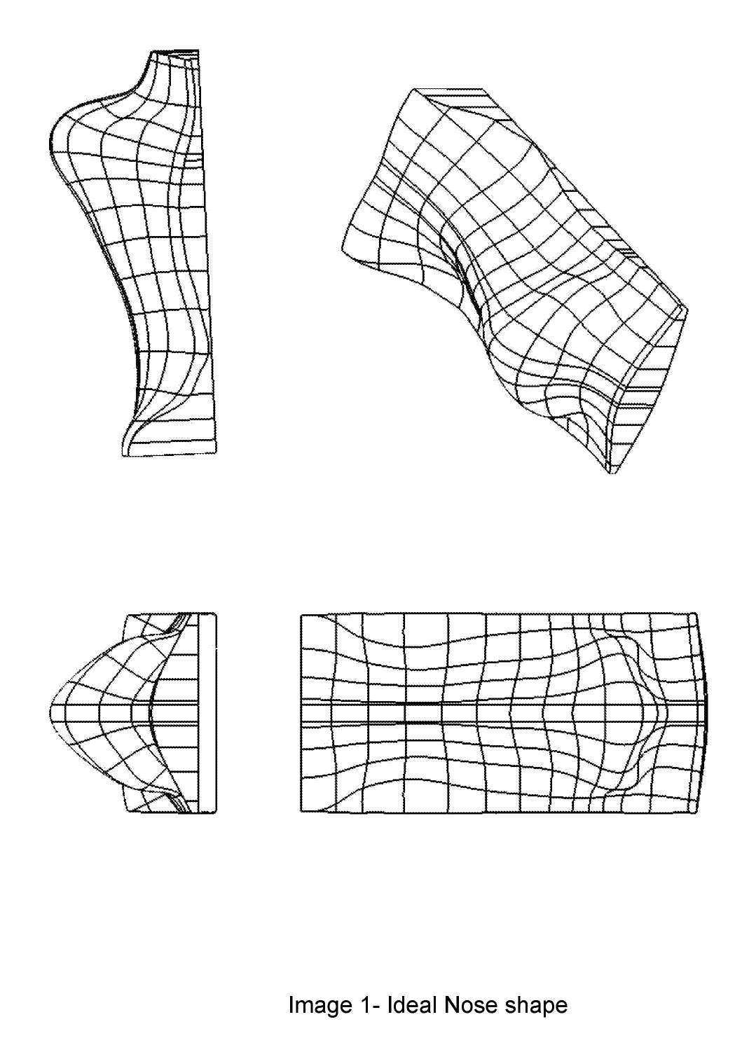

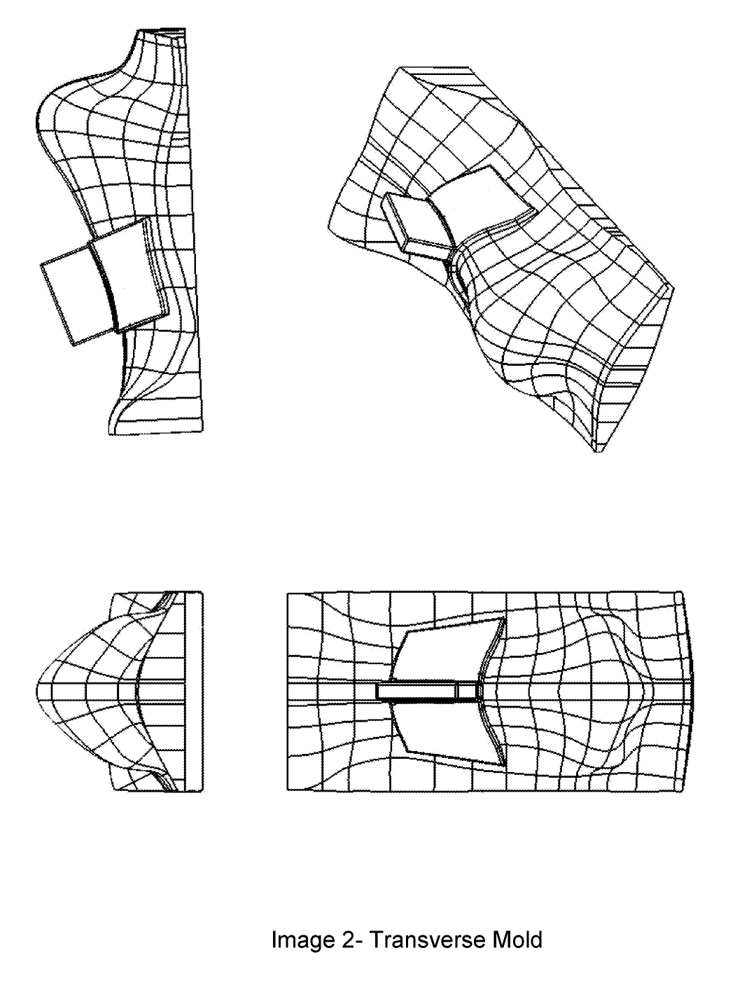

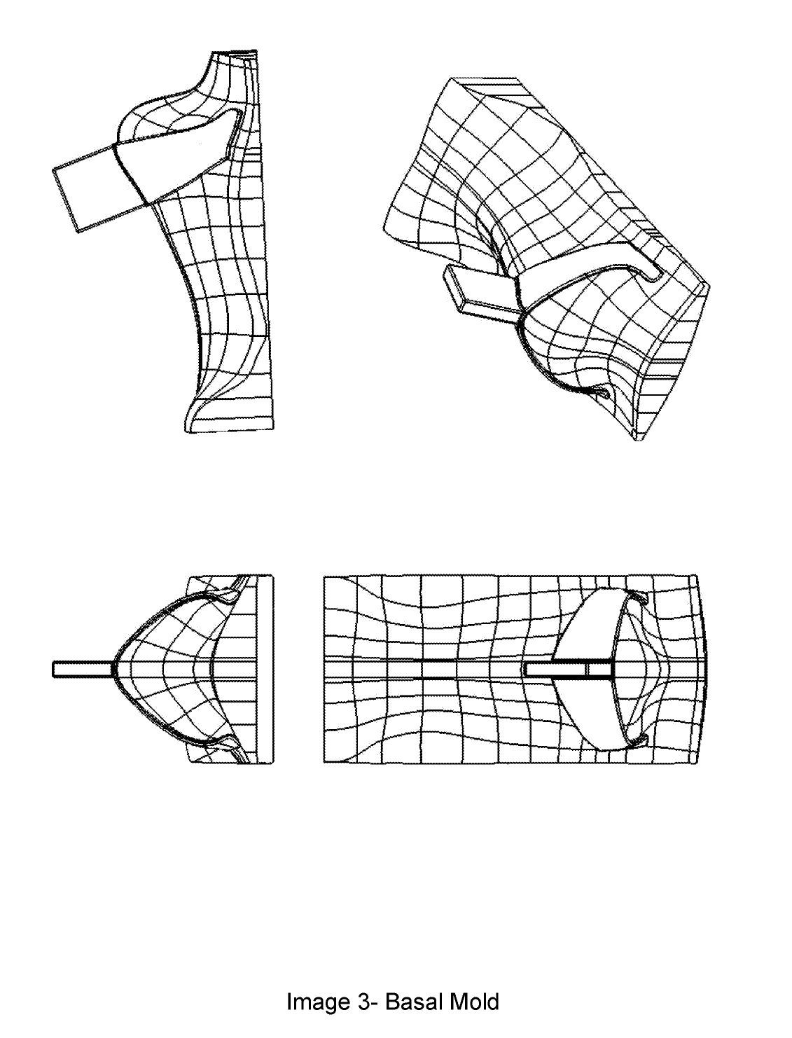

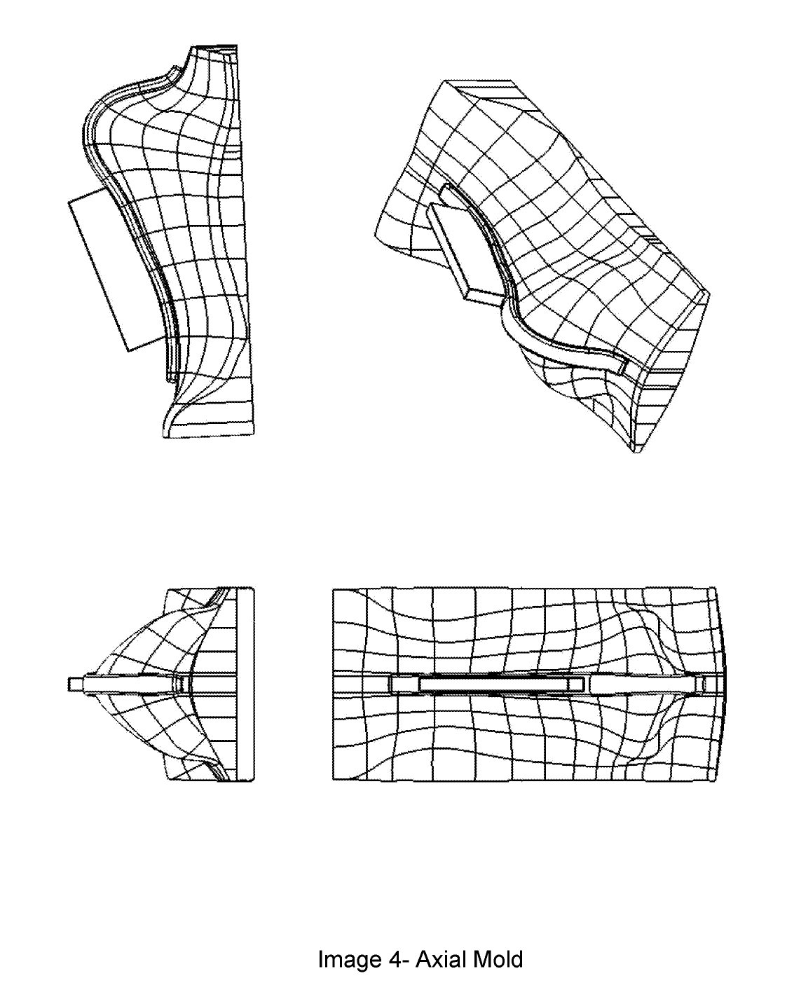

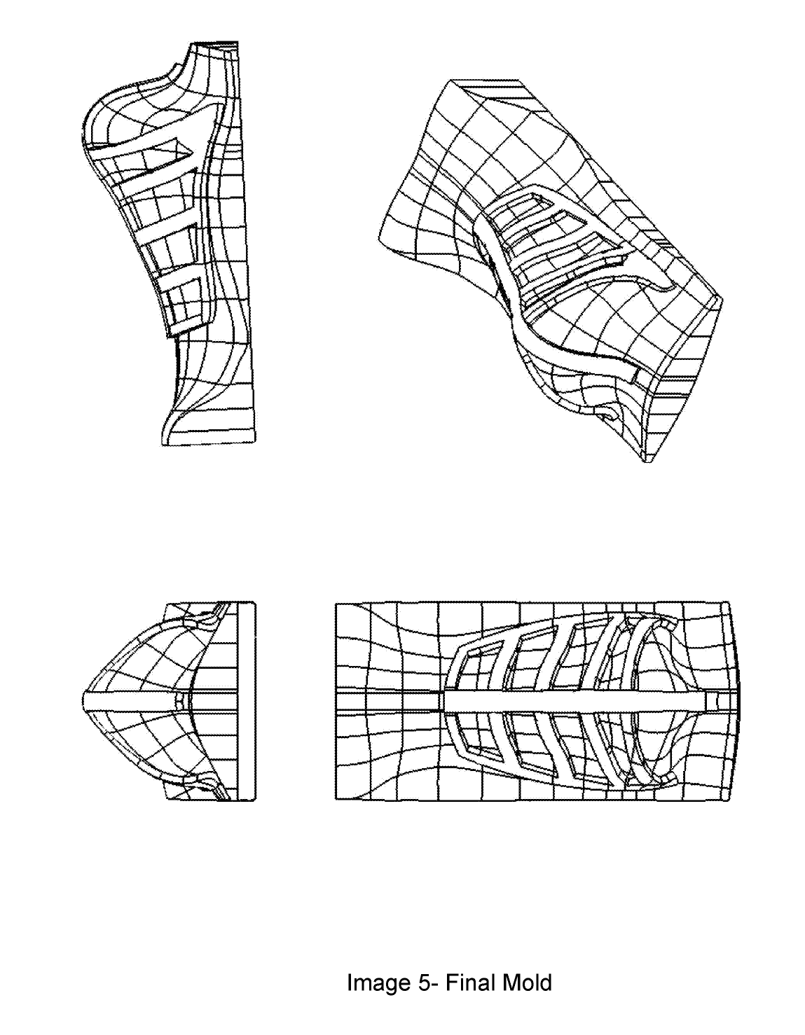

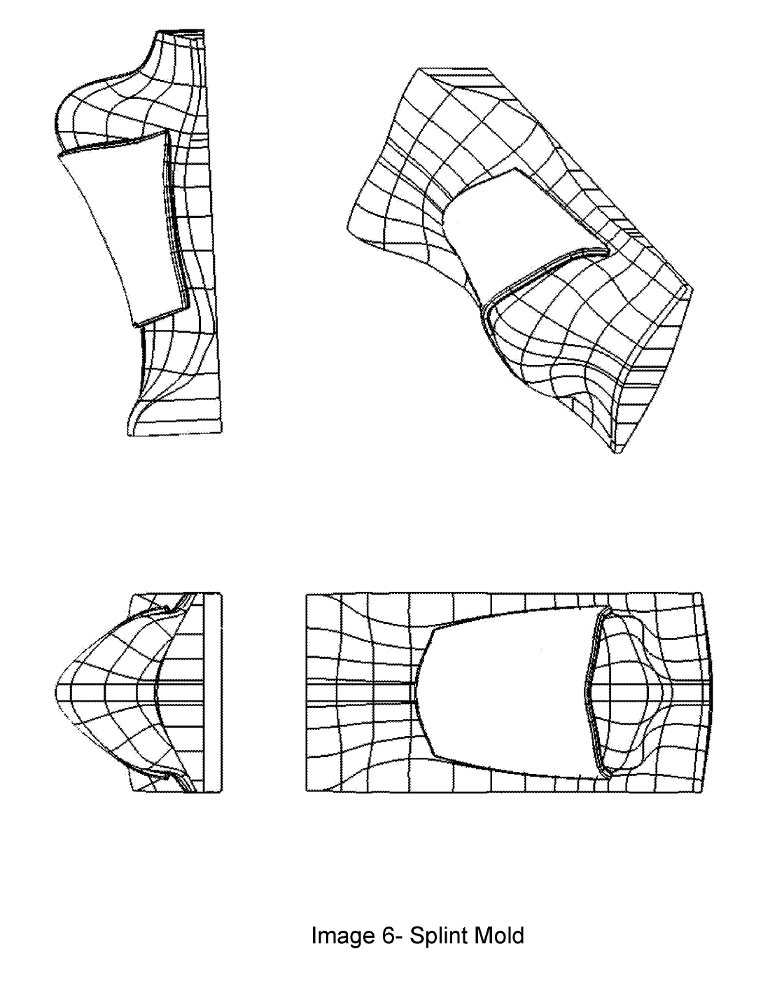

### BACKGROUND OF THE INVENTION

1. Field of the Invention

This invention is used for cosmetic rhinoplasty

2. Description of the Related Surgery

Generally, in rhinoplasty operation, after performing local injections and incisions, skin and soft tissue flap is made by dissecting sub periosteal plane. Therefore, hump removal and dorsal correction either by using osteotom or dorsal rasp should be done. Then septoplasty consequently taking harvest graft are conducted precisely. Reforming of tip by incising and stitching of lower lateral cartilages will be achieved. According to surgeon preference, some cartilaginous grafts may be needed for supporting nose structure such as collumellar strut, spreader and alar grafts. Medial and lateral osteotomy will be done respectedly.

The final part of rhinoplasty is alar correction by creating alar incisions and stitching of them (if needed). At the end of operation, external splint is fixed on nasal skeleton to keep nasal bones in proper position.

All above issues are only visually controlled by surgeon and there is no standard device to examine the correctness of changes and procedure. For that reason different surgeons achieve various results because of different decisions made during surgery by individual surgeon. If surgeon's decisions don't lead applicants to the favorable nose shape, it could end up having unhappy and unsatisfied patients. Traditional rhinoplasty method can increase the risk of having unsatisfied applicants due to high risk of surgeon's errors during the operation.

### BRIEF SUMMARY OF THE INVENTION

### Problem Description

In most rhinoplasty Cases, it is necessary for surgeon to examine the exactitude of the surgery and refine disfigurements. Since surgeons have no instrument to measure changes during the surgery, they have to use their bare eye to decide whether to modify the configuration or not, which can cause the result to be different from what is contemplated by applicant.

I designed 5 custom control molds to control correctness of different operation steps during the surgery to minimize surgeon's errors. Using these control molds enables surgeon to decide whether to keep modifying different part of nose in order to achieve best result.

### Solution

Applicant's current face 3D model can be produced by 3D scanner and then surgeon designs applicant's Ideal nose form. This leads to design and production of control molds to be used in several steps of cosmetic rhinoplasty operation. Surgeon can control the correctness of nasal configuration at the end of each step with molds, since these molds are produced prior to operation, based on the standard nose form, they increase exactitude of operation vastly.

Transverse mold is designed to examine the nasal skeleton location correctness. Axial mold is designed to examine the correctness of tip, dorsum and columella. Basal mold is designed to examine the alar form based on contemplated form.

Final mold is designed to examine the harmony of dorsum, tip, alar and nasal skeleton combination. Splint is designed based on desired nose form.

3D printed molds are delivered to surgeon, sterilized by autoclave or disinfected by Cidex. Operation team controls the applicant's nose after any step of operation by molds.

### BRIEF DESCRIPTION OF DRAWINGS

Image 1—Ideal Nose shape

Image 2—Transverse Mold

Image 3—Basal Mold

Image 4—Axial Mold

Image 5—Final Mold

Image 6—Splint Mold

### DETAILED DESCRIPTION OF THE INVENTION

In most rhinoplasty cases surgeon should control whether different parts of Applicant's nose is formed well and refine disfigurements. Therefore, it is true that in traditional rhinoplasty method, all the decisions are made by surgeon and the only examination method is eye inspection in every step of surgery. Therefore, final result is totally dependent on surgeon's decisions and different surgeons have different results. Further, unfavorable result causes applicant's dissatisfaction. In the other hand many surgeons do not get to picture applicant's contemplated shape. That leads to major difficulties in rhinoplasty for both surgeon and applicants.

Surgeons check the correctness of each step of the surgery by visual inspection and that's why making wrong decisions are probable to be made during surgery. To solve these problems, helping the surgeon and reassuring the patient for achieving the favorable nose shape, I designed a new rhinoplasty method by using the custom control molds. I used a 3D scanner, capable of scanning applicant's face with high quality and produce a 3D model of the applicant's face. Post-surgery form of applicant's nose is designed in 3D by graphical and engineering software. By using the final favorite nose form designed by the software, I made 5 control molds to control each step of surgery. Control molds include:

1. Transverse mold

2. Axial Mold

3. Basal Mold

4. Final Mold

5. Splint Mold

Each control mold is designed to be used in a different stage of rhinoplasty as a guide for surgeon. For example the transverse mold is used after osteotomy as a guide for positioning nasal skeleton correctly and the axial mold is used for checking the form of columella, dorsum and tip. We can put control molds on patient's nose and see the difference between nose parts and the control molds during operation. This method enables surgeon to control the difference between the shape of applicant's nose and the ideal predesigned nose shape in each stage and decide whether to keep refining the nose parts or not. So surgeon and the patients have the same idea of post-surgery nose ideal form.

As edema is in its minimum level right before sutures are applied and operated nose will look most similar to what it will in 6-12 month right then, therefore this is the best time to use the control molds and make sure about the result.

As a result it increases the surgery precision and the surgeon can make more accurate decisions with these control molds.

### PRIOR ART

1. Arima et al,, Crooked nose: outcome evaluations in rhinoplasty, Braz Otorhinolaryngol. 2011.

2. Faidiga et al., Long-Term Evaluation in Aesthetic Rhinoplasty in an Academic Referral Center, Braz J Otorhinolaryngol. 2010.

3. Izu et al., Normal values for the Rhinoplasty Outcome Evaluation (ROE) questionnaire, Braz J Otorhinolaryngol, 2012.

4. Suemi et al., Normal values for the Rhinoplasty Outcome Evaluation (ROE) questionnaire, Braz J Otorhinolaryngol. 2012.

### BOOK

1. Mathes S J and Nahai F, Reconstructive Surgery, Elsevier Health Sciences, 1996

I claim:

1. Rhinoplasty control molds (transverse, basal, axial, final, final splint) reduce surgeon error rate during surgery and are used as a surgical guide during operation, so surgeon will be able to check correctness of the shape of different parts of the nose, which leads to an increase in exactitude and reduces the duration of operation.

2. Applicant's face will be 3D scanned leading to make a 3D model of patient's face.

3. Rhinoplasty control molds claim 2, wherein the created 3D model is imported in designing software and applicants can design their ideal form, in conjunction with surgeon's consultation.

4. Rhinoplasty control molds claim 1, wherein the custom control molds for rhinoplasty are designed based on patient ideal nose shape.

5. Rhinoplasty control molds claim 1, wherein the custom control molds are produced with 3D printer.

6. Rhinoplasty control molds claim 1, wherein the transverse mold is used after osteotomy as a guide to settle nasal skeleton correctly.

7. Rhinoplasty control molds claim 1, wherein the axial mold is used to check the dorsum, tip and columella.

8. Rhinoplasty control molds claim 1, wherein the Basal mold is used to check alar area.

9. Rhinoplasty control molds claim 1, wherein the final mold duty is checking the Dorsum, Tip and alar and basal area combined.

10. Rhinoplasty control molds claim 1, wherein the pre-shaped splint is used to maintain the nose parts in ideal form and reduce the surgery duration. Rhinoplasty control molds claim 1, wherein the control molds could be made of PLA, ABS, PVA, HIPS, PA, CE ABS, PETG, POM, PC materials.

12. Rhinoplasty control molds claim 1, wherein the control molds could be made of transparent materials.

13. Rhinoplasty control molds claim 1, wherein all the control molds could be sterilized before surgery.

14. Rhinoplasty control molds claim 1, wherein all the control molds are used as a surgical guide during operation for different steps of surgery,

15. Rhinoplasty control molds claim 1, wherein rhinoplasty with control molds could reduce the surgery duration and minimize surgeon's errors.
